(19) United States
(12) Patent Application Publication
(10) Pub. No.: US 2019/0163722 A1
(43) Pub. Date: May 30, 2019

(54) FISHER'S EXACT TEST CALCULATION APPARATUS, METHOD, AND PROGRAM

(71) Applicants: NIPPON TELEGRAPH AND TELEPHONE CORPORATION, Chiyoda-ku (JP); TOHOKU UNIVERSITY, Sendai-shi (JP)

(72) Inventors: Satoshi HASEGAWA, Musashino-shi (JP); Koki HAMADA, Musashino-shi (JP); Koji CHIDA, Musashino-shi (JP); Masao NAGASAKI, Sendai-shi (JP); Kazuharu MISAWA, Sendai-shi (JP)

(73) Assignees: NIPPON TELEGRAPH AND TELEPHONE CORPORATION, Chiyoda-ku (JP); TOHOKU UNIVERSITY, Sendai-shi (JP)

(21) Appl. No.: 16/313,344

(22) PCT Filed: Jun. 30, 2017
(86) PCT No.: PCT/JP2017/024128

§ 371 Date: Dec. 26, 2018

(30) Foreign Application Priority Data
Jul. 6, 2016 (JP) 2016-134087

(51) Int. Cl. G06F 17/18; G16B 50/40; G16B 40/00

(52) U.S. Cl. CPC G06F 17/18; G16B 40/00; G16B 50/40

(57) ABSTRACT

A Fisher's exact test calculation apparatus includes a selection unit that selects summary tables for which a result of Fisher's exact test indicative of being significant will be possibly obtained from among a plurality of summary tables based on a parameter obtained in calculation in course of determining the result of Fisher's exact test, and a calculation unit that performs calculations for Fisher's exact test for each of the selected summary tables.

### TECHNICAL FIELD

The present invention relates to techniques for efficiently calculating Fisher's exact test.

### BACKGROUND ART

Fisher's exact test is widely known as one of statistical test methods. An application of Fisher's exact test is genome-wide association study (GWAS) (see Non-patent Literature 1, for instance). Brief description of Fisher's exact test is given below.

TABLE 1

| | X | Y | Total |
|---|---|---|---|
| A | a | b | a + b |
| G | c | d | c + d |
| Total | a + c | b + d | a + b + c + d (=n) |

This table is an example of a 2×2 summary table that classifies n subjects according to character (X or Y) and a particular allele (A or G) and counts the results, where a, b, c, and d represent frequencies (non-negative integers). In Fisher's exact test, when the following is assumed for a non-negative integer i,

$${p}_{i} = \frac{( a + b ) ! ( c + d ) ! ( a + c ) ! ( b + d ) !}{n ! i ! ( a + b - i ) ! ( a + c - i ) ! ( d - a + i ) !}$$

it is determined whether there is a statistically significant association between the character and a particular allele based on the magnitude relationship between:

$$p = {p}_{a} + {{Σ}_{max ( 0 , a - d ) \leq i \leq min ( a + b , a + c )}}_{{p}_{i} < {p}_{a}} {p}_{i}$$

and a threshold T of a predetermined value. In genome-wide association study, a summary table like the above one can be created for each single nucleotide polymorphism (SNP) and Fisher's exact test can be performed on each one of the summary tables. Genome-wide association study involves an enormous number of SNPs on the order of several millions to tens of millions. Thus, in genome-wide association study, there can be a situation where a large quantity of Fisher's exact test is performed.

Meanwhile, in view of the sensitivity or confidentiality of genome information, some prior studies are intended to perform genome-wide association study while concealing genome information via encryption techniques (see Non-patent Literature 2, for instance). Non-patent Literature 2 proposes a method of performing a chi-square test while concealing genome information.

### PRIOR ART LITERATURE

### Non-Patent Literature

Non-patent Literature 1: Konrad Karczewski, “How to do a GWAS”, GENE 210: Genomics and Personalized Medicine, 2015.
Non-patent Literature 2: Yihua Zhang, Marina Blanton, and Ghada Almashaqbeh, “Secure distributed genome analysis for GWAS and sequence comparison computation”, BMC medical informatics and decision making, Vol. 15, No. Suppl 5, p. S4, 2015.

### SUMMARY OF THE INVENTION

### Problems to be Solved by the Invention

Since a single execution of Fisher's exact test requires calculation of a maximum of n/2 types of pᵢ and Fisher's exact test can be conducted on individual ones of a large quantity of summary tables in the case of genome-wide association study in particular, it could involve an enormous processing time depending on the computer environment and/or the frequencies in the summary tables.

An object of the present invention is to provide a Fisher's exact test calculation apparatus, method, and program for performing calculations for multiple executions of Fisher's exact test in a more efficient manner than conventional arts.

### Means to Solve the Problems

A Fisher's exact test calculation apparatus according to an aspect of the present invention includes a selection unit that selects summary tables for which a result of Fisher's exact test indicative of being significant will be possibly obtained from among a plurality of summary tables based on a parameter obtained in calculation in course of determining the result of Fisher's exact test, and a calculation unit that performs calculations for Fisher's exact test for each of the selected summary tables.

### Effects of the Invention

The present invention can perform calculations for multiple executions of Fisher's exact test in a more efficient manner than conventional arts. More specifically, effects such as a reduced usage of calculation resources and/or a shortened processing time are expected to be achieved.

### BRIEF DESCRIPTION OF THE DRAWINGS

FIG. 1 is a block diagram for describing an example of a Fisher's exact test calculation apparatus.

FIG. 2 is a flow diagram for describing an example of a Fisher's exact test calculation method.

### DETAILED DESCRIPTION OF THE EMBODIMENT

An embodiment of the present invention is described below with reference to the drawings.

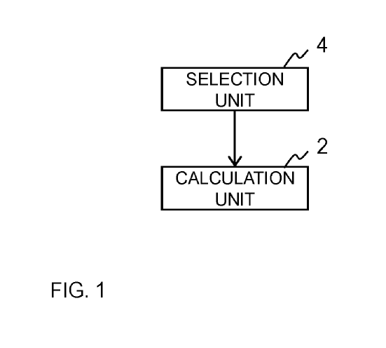

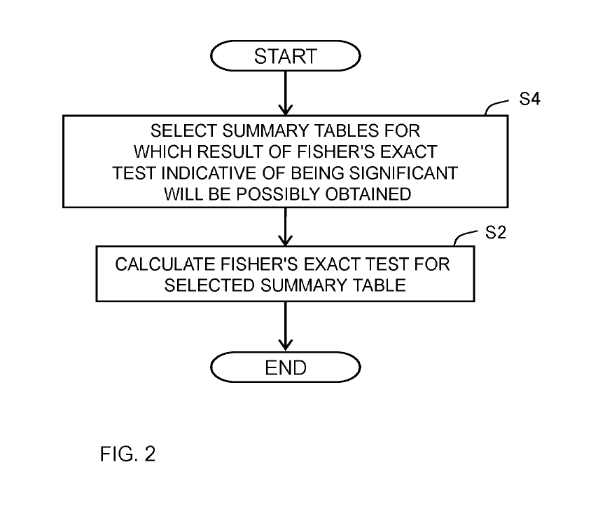

As shown in FIG. 1, a Fisher's exact test calculation apparatus includes a selection unit 4 and a calculation unit 2, for example. A Fisher's exact test calculation method is implemented by these units of the Fisher's exact test calculation apparatus performing the processing at steps S4 and S2 described in FIG. 2 and below.

<Selection Unit 4>

The present Fisher's exact test calculation apparatus and method do not perform calculations for Fisher's exact test for each of in summary tables, where m is a positive integer. Instead, they are given a conditional expression of a sufficient condition under which a result of Fisher's exact test (“TRUE” indicative of having statistically significant association if p is below a threshold T representing a significance level; “FALSE” otherwise) is FALSE, for example. Then, any summary table that does not satisfy this conditional expression, in other words, any summary table for which the result of Fisher's exact test will be certainly FALSE, is discarded. For the discarded summary tables, calculation of the p value is not performed; calculations for Fisher's exact test are performed only for summary tables that have not been discarded, in other words, summary tables for which a result of Fisher's exact test indicative of being significant will be possibly obtained.

To this end, the selection unit 4 first selects summary tables for which a result of Fisher's exact test indicative of being significant will be possibly obtained from multiple summary tables (in summary tables) based on a parameter obtained in calculation in the course of determining the result of Fisher's exact test (step S4). Information on the selected summary tables is output to the calculation unit 2.

An example of a conditional expression for a sufficient condition under which the result of Fisher's exact test will be FALSE is pₐ≥T (or pₐ>T). Here, pₐ represents pᵢ when i=a, and is defined by the formula below:

$${p}_{a} = \frac{( a + b ) ! ( c + d ) ! ( a + c ) ! ( b + d ) !}{n ! a ! b ! c ! d !}$$

From the definition of p, p≥T will always hold when pₐ≥T. Accordingly, pₐ≥T can be said to be a conditional expression for a sufficient condition under which the result of Fisher's exact test will be FALSE.

In a case where the conditional expression for the sufficient condition under which the result of Fisher's exact test will be FALSE is pₐ≥T, the selection unit 4 calculates pₐ based on the frequencies in each summary table, determines whether pₐ≥T, and selects summary tables for which pₐ≥T does not hold in the determination, in other words, those summary tables with pₐ<T.

<Calculation Unit 2>

The calculation unit 2 performs calculations for Fisher's exact test for each of the summary tables selected by the selection unit 4 (step S2). For calculations for Fisher's exact test, any of the existing calculation methods may be employed.

Since a single execution of Fisher's exact test requires calculation of a maximum of n/2 types of pᵢ, the number of calculations is reduced to 2/n at maximum if only the calculation of pₐ has to be done. When n=1000, the number of calculations will be reduced to 1/500. However, as Fisher's exact test needs to be performed for summary tables with pₐ<T, the lower the ratio of the summary table with pₐ<T is, the more convenient it will be. The table below is the result of an actual experiment which was conducted with summary tables of genome data (data publicly available without restriction) registered in the NBDC Human Database (Reference Literature 1) for open publication:

TABLE 2

| | The number of summary | The number of summary |
|---|---|---|
| | tables satisfying | tables satisfying |
| | p < 5.0 × 10⁻⁸ | pₐ < 5.0 × 10⁻⁸ |
| Data 1 | 7 | 13 |
| Data 2 | 101 | 133 |
| Data 3 | 33 | 43 |
| Data 4 | 91 | 104 |

The data utilized in the experiment (data 1 to 4) are given in the table below.

TABLE 3

| | | | Control | The | |
|---|---|---|---|---|---|
| | | Case | group | number | |
| Data | | (# of | (# of | of SNPs | |
| No. | Disease | people) | people) | (M) | Accession No. |
| Data 1 | Cardiac infarction | 1,666 | 3,198 | 455,781 | hum0014.v1.freq.v1 |
| Data 2 | Type 2 diabetes | 9,817 | 6,763 | 552,915 | hum0014.v3.T2DM-1.v1 |
| Data 3 | Type 2 diabetes | 5,645 | 19,420 | 479,088 | hum0014.v3.T2DM-2.v1 |
| Data 4 | Stevens-Johnson | 117 | 691 | 449,205 | hum0029.v1.freq.v1 |
| | syndrome | | | | |

For data 1 as an example, the ratio of summary tables with pₐ<T is as sufficiently small as 13/455781≈0.00285%, and when assuming that the number of calculations for determining p is n/2 times the number of calculations of pₐ, the number of calculations for determining p for all SNPs by a common method will be M×n/2=455781×(1666+3198)/2=1,108,459,392 times the number of calculations of pₐ. In contrast, when the summary tables with pₐ<T are determined and only p's for those summary tables are determined according to the present invention, the number of calculations will be M+L×n/2=455781+13×(1666+3198)/2=519,013 times the number of calculations of pₐ; the number of calculations is as low as about 519,013/1,108,459,392≈1/2135.7, compared to the number of calculations required for determining p's for all SNPs by a common method. Here, M is the number of SNPs and L is the number of summary tables with pₐ<T.
Reference Literature 1: NBDC Human Database, the Internet <URL: http://humandbs.biosciencedbc.jp/>

The data used in the experiment were acquired by the Made-to-order Medicine Realization Project (represented by Yusuke Nakamura, director of the RIKEN Center for Genome Medical Sciences), the Made-to-order Medicine Realization Program (represented by Michiaki Kubo, vice director of the RIKEN Center for Integrative Medical Sciences), and the Frontier Medical Science and Technology for Ophthalmology (represented by Mayumi Ueta, an associate professor of Medical Study Department of Kyoto Prefectural University of Medicine) and provided through the “National Bioscience Database Center (NBDC)” website (http://humandbs.biosciencedbc.jp/) of the Japan Science and Technology Agency (JST).

The calculation unit 2 may also perform calculations for obtaining the result of Fisher's exact test corresponding to the frequencies (a, b, c, d) in the input summary table subjected to Fisher's exact test while keeping the frequencies (a, b, c, d) concealed via secure computation. This secure computation can be carried out with the existing secure computation techniques described in Reference Literatures 2 and 3, for example.
Reference Literature 2: Ivan Damgard, Matthias Fitzi, Eike Kiltz, Jesper Buus Nielsen and Tomas Toft, “Unconditionally secure constant-rounds multi-party computation for equality, comparison, bits and exponentiation”, In Proc. 3rd Theory of Cryptography Conference, TCC 2006, volume 3876 of Lecture Notes in Computer Science, pages 285-304, Berlin, 2006, Springer-Verlag
Reference Literature 3: Takashi Nishide, Kazuo Ohta, “Multiparty Computation for Interval, Equality, and Comparison Without Bit-Decomposition Protocol”, Public Key Cryptography—PKC 2007, 10th International Conference on Practice and Theory in Public-Key Cryptography, 2007, P. 343-360

By thus performing calculations for Fisher's exact test only for summary tables for which a result of Fisher's exact test indicative of being significant will be possibly obtained, in other words, by not performing computation of p for summary tables for which the result of Fisher's exact test will be obviously FALSE, the amount of calculation for Fisher's exact test on multiple summary tables can be decreased.

As to the effect of reduction in computation, since pₐ is calculated as:

$${p}_{a} = \frac{( a + b ) ! ( c + d ) ! ( a + c ) ! ( b + d ) !}{n ! a ! b ! c ! d !}$$

hence,

$$log {p}_{a} = {\sum }_{j = 1}^{a + b} log j + {\sum }_{j = 1}^{c + d} log j + {\sum }_{j = 1}^{a + c} log j + {\sum }_{j = 1}^{b + d} log j - {\sum }_{j = 1}^{n} log j - {\sum }_{j = 1}^{a} log j - {\sum }_{j = 1}^{b} log j - {\sum }_{j = 1}^{c} log j - {\sum }_{j = 1}^{d} log j \qquad Formula A$$

thus, by precomputing log j for k=0, 1, 2, . . . , n and determining log pₐ with the precomputed value, it can be calculated just by additions and subtractions of precomputed values. It may be then determined whether log pₐ>log T. Here, Σⱼ₌₁⁰ log j=0 holds.

[Modifications and Others]

The selection unit 4 may perform the selection of summary tables described above while keeping the frequencies in multiple summary tables concealed via secure computation.

That is, the selection unit 4 may, for example, perform calculations for determining whether the result of Fisher's exact test satisfies the conditional expression for the sufficient condition under which the result will be FALSE, while concealing the input and output.

Such a calculation can be carried out, for example, by precomputing Σⱼ₌₁^{k} log j for k=0, 1, 2, . . . , n and combining encryption techniques capable of magnitude comparison, determination of equality, and addition/subtraction and multiplication while concealing the input and output. In the following, magnitude comparison with the input and output concealed (hereinafter abbreviated as input/output-concealed magnitude comparison) is described. Assume that two values x and y for magnitude comparison are the input and at least one of x and y is encrypted such that its real numerical value is not known. In the present description, only x is encrypted, which is denoted as E(x). The result of magnitude comparison, which is to be output, is defined as:

$$z = \{ \begin{matrix}1 & if x \geq y \\ 0 & otherwise\end{matrix}$$

That is to say, the input/output-concealed magnitude comparison means determining cipher text E(z) for the result of magnitude comparison by using E(x),y as the input and without decrypting E(x). When z is the result to be finally obtained, E(z) is appropriately decrypted. Examples of such input/output-concealed magnitude comparison are the methods described in Reference Literature 2 and 3, for example. Similarly, in the case of determination of equality, z will be z=1 if x=y. Examples of this are also the methods of Reference Literature 2 and 3.

A specific example of calculation of log pₐ in Formula A is given. The input is E(a+b), E(c+d), E(a+c), E(b+d), E(n), E(a), E(b), E(c), E(d), and the output is E(z), where z is 1 when log pₐ>log T, otherwise 0. This will be described for the first term on the right side of Formula A as an example. First, the precomputed value, Σⱼ₌₁^{k} log j (k=0, 1, n), is used to perform secure computation for determination of equality which returns E(1) if a+b=k and E(0) otherwise. Assume that c=1 if a+b=k and c=0 otherwise. Then, by multiplication secure computation, E(cΣⱼ₌₁^{k} log j) is calculated for each k from E(c) and from Σⱼ₌₁^{k} log j.

By finally adding the results while keeping them encrypted, E(Σⱼ₌₁^{a+b} log j) can be obtained. A similar process is then performed for each term on the right side of Formula A and the results are added while being kept encrypted, thus allowing Formula A to be calculated by secure computation.

Assume that the input to the conditional expression for the sufficient condition under which the result of Fisher's exact test will be FALSE is the frequencies, aᵢ, bᵢ, cᵢ, dᵢ, in each summary table i (i=1, 2, . . . , in) and the output is either TRUEᵢ′ or FALSEᵢ′. TRUEᵢ′ or FALSEᵢ′ is denoted as Xᵢ′. The input/output in a concealed state is represented by the symbol E( ). That is to say, aᵢ and TRUEᵢ′, for example, in a concealed state will be represented as E(aᵢ) and E(TRUEᵢ′), respectively. An operation for returning them from a concealed state to the original state (for example, from E(aᵢ) to aᵢ) will be referred to as decryption. Then, the result of whether each summary table satisfies the conditional expression in question, namely Xᵢ′, can give information on the input, aᵢ, bᵢ, cᵢ, dᵢ.

Accordingly, the selection unit 4 may perform the processes of Examples 1 to 3 described below.

### Example 1

The selection unit 4 first determines E(Xᵢ′) from E(aᵢ), E(bᵢ), E(cᵢ), E(dᵢ) using input/output-concealed magnitude comparison, and thereafter randomly shuffles the order of in sets, (E(a₁), E(b₁), E(c₁), E(d₁), E(X₁′), (E(a₂), E(b₂), E(c₂), E(d₂), E(X₂′)), . . . , (E(aₘ), E(bₘ), E(cₘ), E(dₘ), E(Xₘ′)), while concealing the shuffled order. The selection unit 4 then decrypts E(Xᵢ′) and selects summary tables corresponding to sets for which the result of decryption has been TRUEᵢ′.

In this case, the calculation unit 2 performs calculations for Fisher's exact test using E(aᵢ), E(bᵢ), E(cᵢ), E(dᵢ) as the input, in other words, while concealing the input, for the selected summary tables.

With the scheme of Example 1, selection by the selection unit 4 and calculations for Fisher's exact test by the calculation unit 2 can be performed while concealing the frequencies (a, b, c, d) in summary tables for which TRUEᵢ′ has been determined.

### Example 2

In Example 2, the number U of summary tables to be selected is predetermined.

In a similar manner to Example 1, the selection unit 4 calculates E(Xᵢ′) from E(aᵢ), E(bᵢ), E(cᵢ), E(dᵢ) using input/output-concealed magnitude comparison to determine m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)). The selection unit 4 then sorts the m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), while concealing Xᵢ′, such that TRUEᵢ′ is located at the top or the end. For sorting such that TRUEᵢ′ is located at the top or the end, 1 may be set as a flag indicative of TRUEᵢ′ and 0 may be set as a flag indicative of FALSEᵢ′, for example.

The selection unit 4 then selects U sets from the top or the end of the in sets after being sorted. U is a positive integer.

In this case, the calculation unit 2 performs calculations for Fisher's exact test using E(aᵢ), E(bᵢ), E(cᵢ), E(dᵢ) as the input, in other words, while concealing the input, for a selected summary table.

The scheme of Example 2 provides the benefit of enabling further concealment of the number of summary tables for which TRUEᵢ′ has been determined, in addition to the benefit of the scheme of Example 1.

### Example 3

In Example 3, the selection unit 4 first calculates E(Xᵢ′) from E(aᵢ), E(bᵢ), E(cᵢ), E(dᵢ) using input/output-concealed magnitude comparison to determine m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), in a similar manner to Example 1.

The selection unit 4 then probabilistically replaces FALSEᵢ′ with TRUEᵢ′ while concealing them. An exemplary method for probabilistic replacement with TRUEᵢ′ is to prepare in pieces of data, E(Y₁′), E(Y₂′), . . . , E(Yₘ′), for which TRUE′ or FALSE′ is probabilistically concealed in advance, and calculate, for E(Xᵢ′), E(Yᵢ′) (i=1, 2, . . . , m) and while concealing Xᵢ′, Yᵢ′,

$${Z}_{i}^{′} = \{ \begin{matrix}{TRUE}^{′} & if {X}_{i}^{′} = {TRUE}_{i}^{′} OR {Y}_{i}^{′} = {TRUE}^{′} \\ {FALSE}^{′} & Otherwise \end{matrix}$$

The ratio of TRUE′ is appropriately adjusted for Y₁′, Y₂′, . . . , Yₘ′ so that the number of summary tables for which Xᵢ′ will be actually TRUE′ is difficult to infer from the number of summary tables for which Zᵢ′ is TRUE′.

After the replacement, the selection unit 4 performs a similar process to Example 1.

The scheme of Example 3 provides the benefit of enabling further concealment of the number of summary tables for which TRUEᵢ′ has been determined, in addition to the benefit of the scheme of Example 1.

Such concealment enables Fisher's exact test to be executed while concealing genome information and various kinds of associated data, for example. This allows, for example, multiple research institutions to obtain the result of executing Fisher's exact test on combined data while concealing the genome data possessed by the individual institutions and without revealing it to one another, which potentially leads to provision of execution environments for genome analysis of an extremely high security level and hence further development of medicine.

[Program and Recording Medium]

The processes described in connection with the Fisher's exact test calculation apparatus and method may be executed not only in a chronological order in accordance with the order of their description but in a parallel manner or separately depending on the processing ability of the apparatus executing the processes or any necessity.

Also, when the processes of the Fisher's exact test calculation apparatus are to be implemented by a computer, the processing specifics of the functions to be provided by the Fisher's exact test calculation apparatus are described by a program. By the program then being executed by the computer, the processes are embodied on the computer.

The program describing the processing specifics may be recorded on a computer-readable recording medium. The computer-readable recording medium may be any kind of media, such as a magnetic recording device, an optical disk, a magneto-optical recording medium, and semiconductor memory.

Processing means may be configured through execution of a predetermined program on a computer or at least some of the processing specifics thereof may be embodied in hardware.

It will be appreciated that modifications may be made as appropriate without departing from the scope of the present invention.

### INDUSTRIAL APPLICABILITY

As would be apparent from the result of application to genome-wide association study described above, the secure computation techniques of the present invention are applicable to performing Fisher's exact test via secure computation while keeping information on summary tables concealed in an analysis utilizing Fisher's exact test, for example, genome-wide association study, genome analysis, clinical research, social survey, academic study, analysis of experimental results, marketing research, statistical calculations, medical information analysis, customer information analysis, and sales analysis.

1. A Fisher's exact test calculation apparatus comprising:
a selection unit that selects summary tables for which a result of Fisher's exact test indicative of being significant will be possibly obtained from among a plurality of summary tables based on a parameter obtained in calculation in course of determining the result of Fisher's exact test; and
a calculation unit that performs calculations for Fisher's exact test for each of the selected summary tables.

2. The Fisher's exact test calculation apparatus according to claim 1, wherein
where a, b, c, and d represent frequencies in a summary table and T represents significance level, the parameter obtained in calculation in course of determining the result of Fisher's exact test is pₐ defined by the formula below, and
the selection unit selects summary tables with pₐ≤T
$${p}_{a} = \frac{( a + b ) ! ( c + d ) ! ( a + c ) ! ( b + d ) !}{n ! a ! b ! c ! d !} .$$

3. The Fisher's exact test calculation apparatus according to claim 1 or 2, wherein the selection unit performs selection of the summary tables while keeping the frequencies in the plurality of summary tables concealed via secure computation.

4. The Fisher's exact test calculation apparatus according to claim 3, wherein
where m is a positive integer; the plurality of summary tables are a plurality of summary tables i (i=1, 2, . . . , m); the frequencies in the summary table i are represented as aᵢ, bᵢ, cᵢ, dᵢ; information generated by concealing aᵢ, bᵢ, cᵢ, dᵢ is represented as E(aᵢ), E(bᵢ), E(cᵢ), E(dᵢ), respectively; and information indicating whether the summary table i is a summary table for which a result of Fisher's exact test indicative of being significant will be possibly obtained or not is represented as E(Xᵢ′),
the selection unit securely computes E(Xᵢ′) from E(aᵢ), E(bᵢ), E(cᵢ), E(dᵢ) based on the parameter obtained in calculation in course of determining the result of Fisher's exact test so as to determine m sets, (E(a₁), E(b₁), E(c₁), E(d₁) E(X₁′)), (E(a₂), E(b₂), E(c₂), E(d₂), E(X₂′)), . . . , (E(aₘ), E(bₘ), E(cₘ), E(dₘ), E(Xₘ′)), and shuffles an order of the m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), while concealing the shuffled order, decrypts E(Xᵢ′), and selects summary tables for which a result of Fisher's exact test indicating that a result of the decryption is significant will be possibly obtained.

5. The Fisher's exact test calculation apparatus according to claim 3, wherein
where m is a positive integer; the plurality of summary tables are a plurality of summary tables i (i=1, 2, . . . , m); the frequencies in the summary table i are represented as aᵢ, bᵢ, cᵢ, dᵢ; information generated by concealing a₁, bᵢ, cᵢ, dᵢ is represented as E(aᵢ), E(bᵢ), E(cᵢ), E(dᵢ), respectively; information indicating whether the summary table i is a summary table for which a result of Fisher's exact test indicative of being significant will be possibly obtained or not is represented as E(X₁′); and U is a positive integer,
the selection unit securely computes E(Xᵢ′) from E(aᵢ), E(bᵢ), E(cᵢ), E(dᵢ) based on the parameter obtained in calculation in course of determining the result of Fisher's exact test so as to determine m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), sorts the m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), while concealing Xᵢ′ such that the information indicating whether the summary table i is a summary table for which a result of Fisher's exact test indicative of being significant will be possibly obtained or not is located at a top or an end, and selects U sets from the top or the end of the m sets after being sorted.

6. The Fisher's exact test calculation apparatus according to claim 3, wherein
where m is a positive integer; the plurality of summary tables are a plurality of summary tables i (i=1, 2, . . . , m); the frequencies in the summary table i are represented as aᵢ, bᵢ, cᵢ, dᵢ; information generated by concealing aᵢ, bᵢ, cᵢ, dᵢ is represented as E(aᵢ), E(bᵢ), E(cᵢ), E(dᵢ), respectively; and information indicating whether the summary table i is a summary table for which a result of Fisher's exact test indicative of being significant will be possibly obtained or not is represented as E(Xᵢ′),
the selection unit securely computes E(Xᵢ′) from E(aᵢ), E(bᵢ), E(cᵢ), E(dᵢ) based on the parameter obtained in calculation in course of determining the result of Fisher's exact test so as to determine m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), and if Xᵢ′ is information that represents not being a summary table for which a result of Fisher's exact test indicative of being significant will be possibly obtained for at least one set of the m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), replaces that Xᵢ′ with information that represents being a summary table for which a result of Fisher's exact test indicative of being significant will be possibly obtained while concealing the Xᵢ′, shuffles the order of the m sets, (E(a₁), E(b₁), E(c₁), E(d₁), E(X₁′)), (E(a₂), E(b₂), E(c₂), E(d₂), E(X₂′)), . . . , (E(aₘ), E(bₘ), E(cₘ), E(dₘ), E(Xₘ′)), after the replacement while concealing the shuffled order, decrypts E(Xᵢ′), and selects summary tables for which a result of Fisher's exact test indicating that a result of the decryption is significant will be possibly obtained.

7. A Fisher's exact test calculation method comprising:
a selection step in which a selection unit selects summary tables for which a result of Fisher's exact test indicative of being significant will be possibly obtained from among a plurality of summary tables based on a parameter obtained in calculation in course of determining the result of Fisher's exact test; and
a calculation step in which a calculation unit performs calculations for Fisher's exact test for each of the selected summary tables.

8. A non-transitory computer-readable recording medium in which a program for causing a computer to function as the units of the Fisher's exact test calculation apparatus according to claim 1.
